(19) United States
(12) Patent Application Publication
(10) Pub. No.: US 2005/0015113 A1
(43) Pub. Date: Jan. 20, 2005

(54) Guide system for controlled manipulation of surgical instruments

(76) Inventors: Reginald C. Baptiste, Paradise Valley, AZ (US); Scott A. Sher, Scottsdale, AZ (US)

Correspondence Address: Charlene R. Jacobsen;Meschkow & Gresham, PLC, Suite 409, 5727 North Seventh Street, Phoenix, AZ, 85014, US

(21) Appl. No.: 10/622,794

(22) Filed: Jul. 18, 2003

(51) Int. Cl. A61B 17/28

(52) U.S. Cl. 606/205

(57) ABSTRACT

A guide system (20) enables controlled manipulation of a grasping forceps (22) relative to a cutter stapler (24). The guide system (20) includes a body (26) for surrounding a shaft (40) of the stapler (24), with a stapler head (42) and a stapler actuator (44) extending from opposing ends of the body (26). An articulating member (28) is in longitudinal alignment with the body (26) and is selectively moveable to cause an extension portion (64) of the member (28) to move to an extended position (82) transverse from the body. A sleeve (30), coupled to the extension portion (64), retains an elongate flexible member (34) of the grasping forceps (22). Movement of the extension portion (64) causes corresponding movement of the sleeve (30) to adjust a working angle (72) between a grasper end (36) of the grasping forceps (22) and the stapler head (42).

### TECHNICAL FIELD OF THE INVENTION

The present invention relates to the field of surgical instruments for endoscopic procedures. More specifically, the present invention relates to a guide system for controlled manipulation of a grasping forceps relative to a cutter stapler instrument.

### BACKGROUND OF THE INVENTION

For many surgical procedures, the method of choice has shifted from traditional open surgery to the use of less invasive means, sometimes referred to as endoscopic surgery. Endoscopic procedures entail the use of small diameter instruments such as cameras, graspers, forceps, retractors, dissectors, staplers, clamps, and so forth, inserted through small incisions in a body cavity or through cannulas inserted into the body. Endoscopic surgical procedures utilizing these small diameter instruments usually result in less pain, scarring, and recovery time for the patient, and a resultant reduction in health-care costs.

An endoscopic surgical procedure to excise tissue for biopsy and/or to remove diseased tissue typically calls for the concurrent insertion and manipulation of two or more surgical instruments through two or more openings. These openings are conventionally created by piercing the body using a trocar. Once the trocar has pierced the skin and its tip is pushed inside the body, the surgeon pulls out the piercing component trocar, leaving only the hollow trocar.

During an endoscopic procedure to remove lung tissue, for example, a grasping forceps may be inserted through one cannula in the body to manipulate the lung tissue, and a linear cutter stapler may be inserted through another cannula to cut the lung tissue and simultaneously place rows of staples along both sides of the incision in order to prevent excessive bleeding. An endoscope may be inserted through yet another cannula to visualize the surgical site.

Although endoscopic procedures have many advantages, there also exists some important disadvantages. The above example underscores these disadvantages. For the patient, the multiple openings increase scarring, pain, and potential for infection. In addition, the initial entry is fraught with danger. This danger is due to the sudden change in resistance that occurs at the instant when the trocar's tip pierces the body. If the trocar should suddenly penetrate the body too deep, underlying tissues and/or organs may be nicked, thus necessitating an unplanned for open surgery to repair the damaged tissue. Accordingly, the creation of multiple openings exacerbates this danger.

Even when the trocars safely pierce the body, further problems can arise for the surgeon. These problems include: restricted vision, no tactile perception, difficulty in handling the instruments, restricted mobility, and difficult hand-eye coordination. The manipulation of multiple instruments through multiple openings is time consuming and tedious. Moreover, it is difficult to appropriately grasp the tissue and only remove the desired amount due to the angle of attack of each of the instruments relative to one another. Consequently, during such a surgery, the incision may not be straight, but rather curved, resulting in a longer incision and a greater quantity of tissue excised than is desired.

Thus, what is needed is a device for facilitating the cooperative manipulation of a pair of endoscopic surgical instruments, while minimizing the number of openings required to perform an endoscopic surgical procedure.

### SUMMARY OF THE INVENTION

Accordingly, it is an advantage of the present invention that a guide system is provided for controlled manipulation at a surgical site of a first instrument relative to a second instrument.

It is another advantage of the present invention that a guide system is provided for use with a pair of instruments that minimizes a number of openings required to perform an endoscopic surgical procedure.

Another advantage of the present invention is that a guide system is provided for use with a pair of instruments that can be readily manipulated by a surgeon.

Yet another advantage of the present invention is that a guide system is provided that maintains an appropriate working angle between a linear cutter stapler and a grasping forceps when excising tissue during an endoscopic surgical procedure.

The above and other advantages of the present invention are carried out in one form by a guide system for controlled manipulation at a surgical site of a first instrument relative to a second instrument. The first instrument includes an elongate flexible member interposed between a first effector and a first actuator, and the second instrument includes a shaft interposed between a second effector and a second actuator. The guide system includes a body having a first end and a second end. The body is configured to surround the shaft of the second surgical instrument such that the second effector extends from the first end and the second actuator extends from the second end. An articulating member is in longitudinal alignment with the body and is selectively moveable to cause an extension portion of the articulating member to move to an extended position transverse from the body. A sleeve is coupled to the extension portion and is configured to retain the elongate flexible member such that movement of the extension portion causes corresponding movement of the sleeve to adjust a working angle between the first and second effectors.

The above and other advantages of the present invention are carried out in another form by a forceps system for use in cooperation with a cutter stapler. The cutter stapler includes a stapler head, a stapler actuator, and a shaft interposed between the stapler head and the stapler actuator. The forceps system comprises a grasping forceps including a grasper, a grasper actuator, and an elongate flexible member interposed between the grasper and the grasper actuator. The forceps system further comprises a forceps guide system for controlled manipulation of the grasper relative to the stapler head. The guide system includes a body having a distal end and a proximal end. The body is configured to surround the shaft of the cutter stapler such that the stapler head extends from the distal end and the stapler actuator extends from the proximal end. An articulating member is in longitudinal alignment with the body. The articulating member has an activator portion and an extension portion pivotally coupled to the activator portion such that a pushing force applied to the activator portion causes the extension portion to exit from the slot and move to an extended position transverse from the body, and a pulling force applied to the activator portion causes the extension portion to move to a retracted position. A sleeve is coupled to the extension portion and retains the elongate flexible member such that movement of the extension portion causes corresponding movement of the sleeve to adjust a working angle between the grasper and the stapler head.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures, and:

FIG. 1 shows a perspective view of a guide system for controlled manipulation of a pair of surgical instruments in accordance with a preferred embodiment of the present invention;

FIG. 2 shows a side perspective view of the guide system of FIG. 1 retaining a grasping forceps;

FIG. 3 shows an illustrative side view of the guide system in use with a linear cutter stapler;

FIG. 4 shows a side view of the guide system in a retracted position;

FIG. 5 shows a side view of an articulating member of the guide system of FIG. 1 in the retracted position;

FIG. 6 shows a side view of the articulating member of FIG. 5 in a partially extended position;

FIG. 7 shows a side view of the articulating member of FIG. 5 in a fully extended position;

FIG. 8 shows a sectional view of the body of the guide system including the linear cutter stapler along section line 8-8 shown in FIG. 3;

FIG. 9 shows a perspective view of a forceps system in accordance with an alternative embodiment of the present invention; and

FIG. 10 shows a side view of a patient undergoing an exemplary endoscopic surgical procedure using the guide system for controlled manipulation of the grasping forceps relative to the linear cutter stapler.

### DETAILED DESCRIPTION OF THE PREFERRED EMBODIMENTS

The present invention is described in connection with endoscopic surgical procedures and apparatus. However, the use of the term “endoscopic” used herein should not be construed to limit the present invention to instruments for use only in conjunction with an endoscopic tube. Rather, the present invention may find use in any procedure where access is limited to a small incision, including, but not limited to endoscopic and/or laparoscopic procedures.

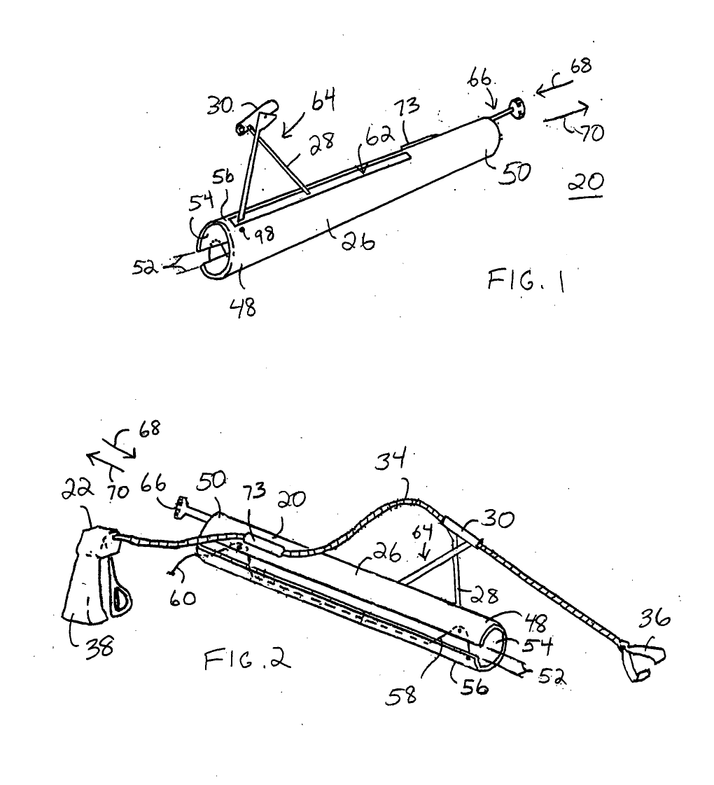

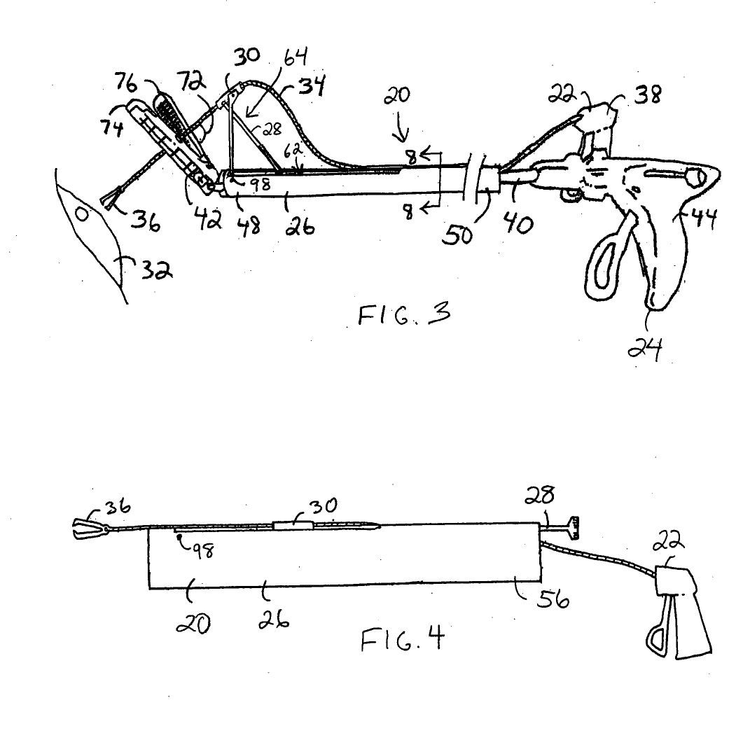

FIG. 1 shows a perspective view of a guide system 20 for controlled manipulation of a pair of surgical instruments in accordance with a preferred embodiment of the present invention. FIG. 2 shows a side perspective view of guide system 20 retaining a grasping forceps 22, and FIG. 3 shows an illustrative side view of guide system 20 in use with a linear cutter stapler 24. Guide system 20 includes a body 26, an articulating member 28 in longitudinal alignment with body 26, and a sleeve 30 coupled to articulating member 28.

Guide system 20 is configured for controlled manipulation at a surgical site 32 of grasping forceps 22 relative to cutter stapler 24. Guide system 20, grasping forceps 22, and cutter stapler 24 are desirably suited for use during an endoscopic surgical procedure. To that end, grasping forceps 22 includes an elongate flexible member 34 interposed between a first effector, i.e., a grasper 36, and a grasper actuator 38. Grasping forceps 22 may include a control wire (not shown) axially movably inserted in flexible member 34. The control wire is advanced or retracted at a control part (not shown) connected to grasper actuator 38, thereby opening or closing grasper 36.

Linear cutter stapler 24 includes a shaft 40 interposed between a second effector, i.e., a stapler head 42, and a stapler actuator 44. Stapler actuator 44 is interconnected with stapler head 42 via shaft 40 and remotely actuates stapler head 42, thus permitting cutter stapler 24 to be remotely operated by the surgeon. In a preferred scenario, stapler head 42 may be articulated via stapler actuator 44 to a forty five degree angle relative to the longitudinal axis of shaft 40 to increase its range of operability. Stapler head 42 desirably applies a double row of staples (not shown) on each side of an incision into surgical site 32. A knife (not shown) at stapler head 42 moves between the double row of staples to longitudinally cut the tissue between the rows of staples.

Body 26 of guide system 20 includes a first end 48 and a second end 50. Body 26 is configured to surround shaft 40 of cutter stapler 24 such that stapler head 42 extends from first end 48 and stapler actuator 44 extends from second end 50. Body 26 optionally includes a gap 52 extending from an interior surface 54 to an exterior surface 56 of body 26 for accommodating installation of shaft 40. Guide system 20 further includes means for closing gap 52 following installation of shaft 40. In an exemplary embodiment, gap 52 may be closed using a cable tensioning system 58 by pulling on and securing a cable tensor end 60 of cable tensioning system 58.

Body 26 includes a longitudinally oriented slot 62, and articulating member 28 includes an extension portion 64 and an activator portion 66 residing within body 26. In general, extension portion 64 is pivotally coupled to activator portion 66. Accordingly, a pushing force, as illustrated by a first arrow 68, applied to extension portion 66 causes extension portion 64 to extend from slot 62 to an extended position transverse from body 26. Conversely, a pulling force, as illustrated by a second arrow 70, applied to activator portion 66 causes extension portion 64 to retract into body 26.

Sleeve 30 is configured to slidably retain flexible member 34. That is, an inner passage of sleeve 30 through which flexible member 34 is routed is larger than an outer diameter of flexible member 34 so that flexible member 34 can freely slide fore and aft in sleeve 30, discussed below.

A guide sleeve 73 is optionally positioned on exterior surface 56 of body 26. Like sleeve 30, guide sleeve 73 is configured to slidably retain flexible member 34 so that flexible member 34 can freely move fore and aft, while still being held close to body 26. Although only one guide sleeve 73 is shown, body 26 may include several aligned guide sleeves for retaining flexible member 34. Alternatively, guide sleeve 73 may extend the length of body 26 from slot 62 to second end 50.

Movement of extension portion 64 causes corresponding movement of sleeve 30 to adjust a working angle 72 between grasper 36 and stapler head 42. As best shown in FIG. 3, working angle 72 is approximately ninety degrees. That is, grasper 36 of grasping forceps 22 is oriented substantially perpendicular to stapler head 42. In such a manner, grasping forceps 22 can be slid forward between first and second jaws 74 and 76, respectively, of stapler head 42 toward surgical site 32. The surgeon can then actuate grasper 36 via grasper actuator 38. Once tissue at surgical site 32 is seized the surgeon withdraws grasping forceps 22 to move surgical site 32 between first and second jaws 74 and 76.

FIG. 4 shows a side view of guide system 20 in a retracted position. That is, articulating member 28 is straight, thus, retracted into body 26. Accordingly, grasping forceps 22 lie relatively flat along exterior surface 56 of body 26. In such a manner, guide system 20 carrying grasping forceps 22 and cutter stapler 24 (FIG. 3) may be readily inserted through a cannula 78 (shown in FIG. 10) into the interior of the body.

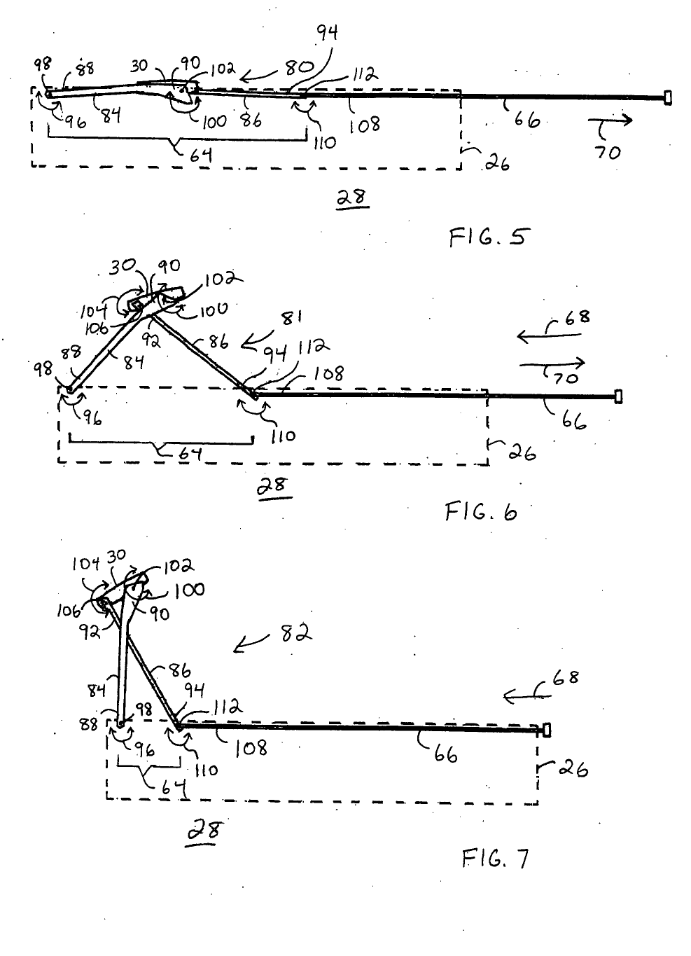

Referring to FIGS. 5-7, FIG. 5 shows a side view of articulating member 28 of guide system 20 (FIG. 1) in a retracted position 80. FIG. 6 shows a side view of articulating member 28 in a partially extended position 81, and FIG. 7 shows a side view of articulating member in a fully extended position 82. Body 26 of guide system 20 (FIG. 1) is shown in ghost form in each of FIGS. 5-7 for simplicity of illustration. Articulating member 28 includes extension portion 64 and activator portion 66. Extension portion 64 includes a first section 84 and a second section 86. First section 84 includes a first end 88 and a second end 90. Similarly, second section 86 includes a third end 92 and a fourth end 94.

First end 88 includes a first pivot joint 96, as represented by a first dual directional arrow. First pivot joint 96 is established by directing a first pivot pin 98 through first end 88 of first section 84 and securing first pivot pin 98 to opposing sides of body 26 to prevent extension portion 64 from translational movement within body 28. First pivot pin 98 serves as a pivot about which first section 84 is able to rotate.

Second end 90 includes a second pivot joint 100, as represented by a second dual directional arrow. Second pivot joint 100 is formed by directing a second pivot pin 102 through second end 90 of first section 84 and securing second pivot pin 102 to sleeve 30. Second pivot pin 102 serves as a pivot about which first section 84 is also able to rotate.

Similarly, third end 92 of second section 86 includes a third pivot joint 104, as represented by a third dual directional arrow. Third pivot joint 104 is formed by directing a third pivot pin 106 through third end 92 and also securing third pivot pin 104 to sleeve 30. Third pivot pin 106 serves as a pivot about which second section 86 is able to rotate.

Additionally, fourth end 94 of second section 86 couples with an activator end 108 of activator portion 66 to establish a fourth pivot joint 110, represented by a fourth dual directional arrow. Fourth pivot joint 110 is established by directing a fourth pivot pin 112 through each of fourth end 94 and activator end 108 of activator portion 66. Fourth pivot pin 112 serves as a pivot about which second section 86 is able to rotate.

As shown, second and third pivot joints 100 and 104, respectively, are located at opposing ends of sleeve 30 and cross one another but do not interconnect. This configuration enables cooperative movement at each of first, second, third and fourth pivot joints 100, 104, 106, and 110, respectively, to adjust sleeve 30 to the appropriate working angle 72 (FIG. 3) as articulating member 28 is actuated to fully extended position 82.

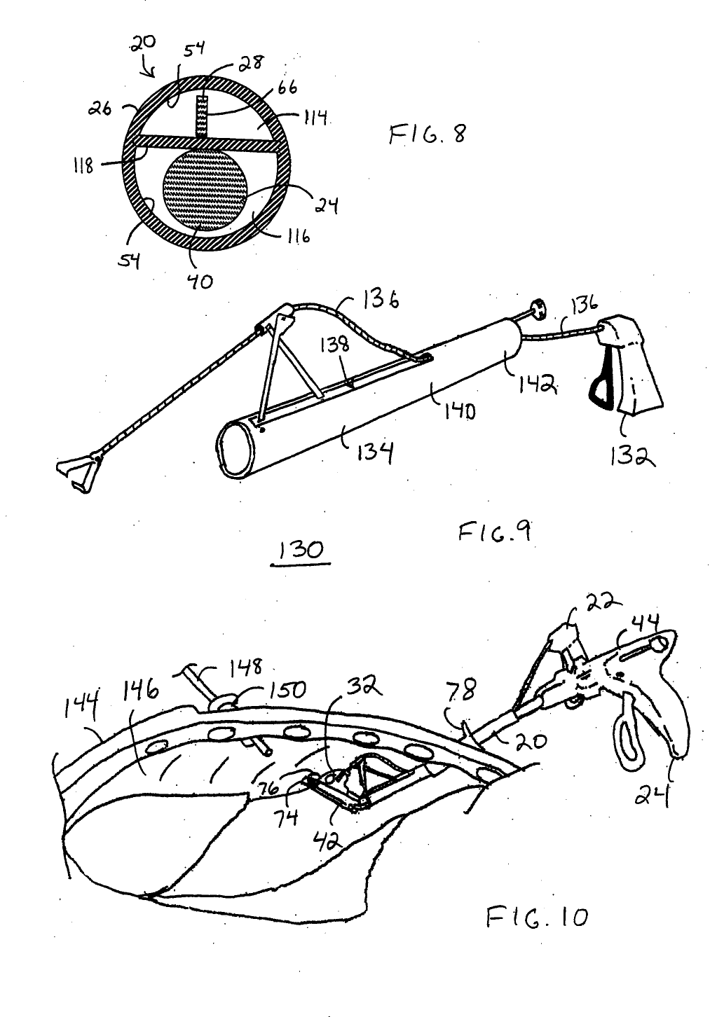

FIG. 8 shows a sectional view of body 26 of guide system 20 including linear cutter stapler 24, along section line 8-8 shown in FIG. 3. Flexible member 34 (FIG. 2) of grasping forceps 22 (FIG. 2) is excluded from FIG. 8 for simplicity of illustration.

In a preferred embodiment, body 26 may include a first passage 114 for location of articulating member 28, and a second passage 116 configured to accommodate shaft 40 of cutter stapler 24. First and second passages 114 and 116, respectively, are defined by interior surface 54 of body 26 and a dividing wall 118. Dividing wall 118 prevents the translational movement of shaft 40 of cutter stapler 24 from interfering with the translational movement of articulating member 28, and vice versa.

In a preferred embodiment, second passage 116 may be sized large enough so that stapler head 42 can readily slide within second passage 116. As such, gap 52 (FIG. 1) may not be necessary for accommodating the installation of shaft 40. Rather, stapler head 42 may be slid through second passage 116 of body 26, after body 26 has been inserted into the patient through cannula 78 (FIG. 10). Accordingly, stapler head 42 may be withdrawn from the patient via second passage 116 so that it can be readily reloaded with staples (not shown) should the need arise during an endoscopic surgical procedure.

FIG. 9 shows a perspective view of a forceps system 130 in accordance with an alternative embodiment of the present invention. Forceps system 130 includes a grasping forceps 132 and a forceps guide system 134. Grasping forceps 132 is similar to grasping forceps 22 (FIG. 2) and forceps guide system 134 is similar to guide system 20. Thus, forceps system 130 integrates grasping forceps 22 and guide system 20 into a single functional unit that may be utilized in cooperation with cutter stapler 24 (FIG. 3). Consequently, the above provided description of grasping forceps 22 and guide system 20 is applicable to grasping forceps 132 and forceps guide system 134 with one notable exception.

Referring momentarily to FIG. 2, it should be recalled that guide system 20 includes guide sleeve 73 positioned on exterior surface 56 of body 26 for slidably retaining flexible member 34 of grasping forceps 22. With reference back to FIG. 9, in contrast to guide system 20, guide system 134 does not include guide sleeve 73. Rather, an elongate flexible member 136 of grasping forceps 132 is directed through a longitudinally oriented slot 138 into a body 140 of forceps guide system 134. Body 140 surrounds flexible member 136 so that forceps system 130 is more readily inserted through cannula 78 (FIG. 10) into the patient. Flexible member 136 subsequently exits from a proximal end 142 of body 140.

FIG. 10 shows a side view of a patient 144 undergoing an exemplary endoscopic surgical procedure using guide system 20 for controlled manipulation of grasping forceps 22 relative to linear cutter stapler 24. Guide system 20 is directed through cannula 78 into the thoracic cavity 146 of patient 144. An endoscope 148 is directed through a second cannula 150 for visualizing surgical site 32.

As discussed in connection with FIG. 3, grasping forceps 22 is slid forward between first and second jaws 74 and 76, respectively, of stapler head 42 toward surgical site 32. The surgeon then actuates grasper 36 via grasper actuator 38 to seize tissue, in this case lung tissue, at surgical site 32. Once the tissue is seized, the surgeon withdraws grasping forceps 22 to move surgical site 32 between first and second jaws 74 and 76. Stapler head 42 of linear cutter stapler 24 is then actuated via stapler actuator 44 to cut the lung tissue at surgical site 32 and simultaneously place rows of staples along both sides of the incision in order to prevent excessive bleeding. The excised tissue can simply be removed from thoracic cavity 146 via cannula 78 when guide system 20 is withdrawn from thoracic cavity 146.

In summary, the present invention teaches of a guide system for controlled manipulation at a surgical site of a grasping forceps relative to a cutter stapler. The guide system enables the concurrent use of both the grasping forceps and the cutter stapler through a single opening during an endoscopic surgical procedure. Since the number of required openings into the patient is reduced, the scarring, pain, and potential for infection are also reduced. Moreover, less openings also result in less potential for damage to underlying organs from sudden penetration of the trocar into the patient. In addition, the guide system retains the grasping forceps and the cutter stapler at an appropriate working angle relative to one another so that a surgeon can readily create a straight incision at a surgical site within a body cavity and only remove the desired amount of tissue.

Although the preferred embodiments of the invention have been illustrated and described in detail, it will be readily apparent to those skilled in the art that various modifications may be made therein without departing from the spirit of the invention or from the scope of the appended claims. For example, although the present invention is described in connection with controlled manipulation of grasping forceps relative to a cutter stapler, it should become apparent to those skilled in the art that the present invention may be adapted for use with other cooperating pairs of endoscopic surgical instruments, such as biopsy forceps, retractors, dissectors, clamps, and so forth.

1. A guide system for controlled manipulation at a surgical site of a first instrument relative to a second instrument, said first instrument including an elongate flexible member interposed between a first effector and a first actuator, and said second instrument including a shaft interposed between a second effector and a second actuator, said guide system comprising:
a body including a first end and a second end, said body being configured to surround said shaft of said second surgical instrument such that said second effector extends from said first end and said second actuator extends from said second end;
an articulating member in longitudinal alignment with said body and selectively moveable to cause an extension portion of said articulating member to move to an extended position transverse from said body; and
a sleeve coupled to said extension portion and configured to retain said elongate flexible member such that movement of said extension portion causes corresponding movement of said sleeve to adjust a working angle between said first and second effectors.

2. A guide system as claimed in claim 1 wherein:
said articulating member resides within said body; and
said body further comprises a longitudinally oriented slot, said extension portion extending from said slot in said extended position.

3. A guide system as claimed in claim 2 wherein said body is further configured to surround said flexible member.

4. A guide system as claimed in claim 2 wherein said body comprises:
a first passage for location of said articulating member; and
a second passage configured to accommodate said shaft.

5. A guide system as claimed in claim 1 wherein said body includes a gap extending from an interior surface to an exterior surface of said body for accommodating installation of said shaft into said body.

6. A guide system as claimed in claim 5 further comprising means for closing said gap following installation of said shaft.

7. A guide system as claimed in claim 1 further comprising a guide sleeve positioned on an exterior surface of said body and configured to slidably retain said flexible member.

8. A guide system as claimed in claim 1 wherein said articulating member further comprises an activator portion, said extension portion being pivotally coupled to said activator portion, wherein a pushing force applied to said activator portion causes said extension portion to move to said extended position, and a pulling force applied to said activator portion causes said extension portion to move to a retracted position.

9. A guide system as claimed in claim 8 wherein said extension portion comprises:
a first section having a first end and a second end, said first end including a first pivot joint, said second end being coupled with said sleeve at a second pivot joint, said first section being able to pivot at each of said first and second pivot joints; and
a second section having a third end and a fourth end, said third end being coupled with said sleeve at a third pivot joint, said fourth end and said activator portion establishing a fourth pivot joint, and said second section being able to pivot at each of said third and fourth pivot joints.

10. A guide system as claimed in claim 1 wherein said sleeve is configured to slidably retain said flexible member.

11. A guide system as claimed in claim 1 wherein a first longitudinal axis of said sleeve is approximately aligned with a second longitudinal axis of said articulating member when said articulating member is in a retracted position.

12. A guide system for controlled manipulation at a surgical site of a forceps relative to a cutter stapler, said forceps including an elongate flexible member interposed between a grasper head and a grasper actuator, and said cutter stapler including a shaft interposed between a stapler head and a stapler actuator, said guide system comprising:
a body including a first end, a second end, and a longitudinally oriented slot, said body being configured to surround said shaft such that said stapler head extends from said first end and said stapler actuator extends from said second end;
an articulating member residing within, and in longitudinal alignment with, said body, said articulating member including an activator portion and an extension portion pivotally coupled to said activator portion, and a pushing force applied to said activator portion causes said extension portion to exit from said slot and move to an extended position transverse from said body, and a pulling force applied to said activator portion causes said extension portion to move to a retracted position within said body; and
a sleeve coupled to said extension portion and configured to retain said elongate flexible member such that movement of said extension portion causes corresponding movement of said sleeve to adjust a working angle between said grasper head and said stapler head.

13. A guide system as claimed in claim 12 wherein said body comprises:
a first passage for location of said articulating member; and
a second passage configured to accommodate said shaft.

14. A guide system as claimed in claim 12 wherein said extension portion comprises:
a first section having a first end and a second end, said first end including a first pivot joint, said second end being coupled with said sleeve at a second pivot joint, said first section pivoting at each of said first and second pivot joints; and
a second section having a third end and a fourth end, said third end being coupled with said sleeve at a third pivot joint, said fourth end and said activator portion establishing a fourth pivot joint, and said second section pivoting at each of said third and fourth pivot joints.

15. A guide system as claimed in claim 12 wherein said sleeve is configured to slidably retain said flexible member.

16. A guide system as claimed in claim 12 wherein a first longitudinal axis of said sleeve is substantially aligned with a second longitudinal axis of said articulating member when said articulating member is in said retracted position.

17. A forceps system for use in cooperation with a cutter stapler, said cutter stapler including a stapler head, a stapler actuator, and a shaft interposed between said stapler head and said stapler actuator, said forceps system comprising:
a grasping forceps including a grasper, a grasper actuator, and an elongate flexible member interposed between said grasper and said grasper actuator; and
a forceps guide system for controlled manipulation of said grasper relative to said stapler head, said guide system including:
a body having a distal end and a proximal end, said body being configured to surround said shaft of said cutter stapler such that said stapler head extends from said distal end and said stapler actuator extends from said proximal end;
an articulating member in longitudinal alignment with said body, said articulating member having an activator portion and an extension portion pivotally coupled to said activator portion, such that a pushing force applied to said activator portion causes said extension portion to exit from said slot and move to an extended position transverse from said body, and a pulling force applied to said activator portion causes said extension portion to move to a retracted position; and
a sleeve coupled to said extension portion and retaining said elongate flexible member such that movement of said extension portion causes corresponding movement of said sleeve to adjust a working angle between said grasper and said stapler head.

18. A forceps system as claimed in claim 17 wherein said sleeve slidably retains said flexible member.

19. A forceps system as claimed in claim 17 further comprising a guide sleeve positioned on an exterior surface of said body and slidably retaining said flexible member.

20. A forceps system as claimed in claim 17 wherein said body surrounds and slidably retains said flexible member.
